(19) United States
(12) Patent Application Publication
(10) Pub. No.: US 2024/0255519 A1
(43) Pub. Date: Aug. 1, 2024

(54) ASYMMETRICAL FLOW FIELD-FLOW FRACTIONATION WITH MASS SPECTROMETRY FOR BIOMACROMOLECULE ANALYSIS

(71) Applicant: Regeneron Pharmaceuticals, Inc., Tarrytown, NY (US)

(72) Inventors: Yuetian Yan, Chappaqua, NY (US); Tao Xing, White Plains, NY (US); Shunhai Wang, Scarsdale, NY (US); Ning Li, New Canaan, CT (US)

(21) Appl. No.: 18/429,572

(22) Filed: Feb. 1, 2024

Related U.S. Application Data
(60) Provisional application No. 63/442,594, filed on Feb. 1, 2023.

(51) Int. Cl. G01N 33/68; H01J 49/16

(52) U.S. Cl. CPC G01N 33/6851; G01N 33/6854; H01J 49/165

(57) ABSTRACT

The present inventions provide new and improved systems and methods combining asymmetrical flow field-flow fractionation (A4F, and also referred to as AF4) and mass spectrometry (MS) in order to analyze for biomacromolecules, including but not limited to Fc-antibodies, antibody fragments, fusion proteins, Fc-fusion proteins, receptor Fc-fusion proteins, trap proteins and mini-trap proteins. Nanoparticles also can be analyzed.

This application claims priority to U.S. Application Ser. No. 63/442,594, filed Feb. 1, 2023, which is hereby incorporated by reference.

### FIELD OF THE INVENTIONS

The present inventions provide new and improved systems and methods combining asymmetrical flow field-flow fractionation (A4F, and also referred to as AF4) and mass spectrometry (MS) in order to analyze for biomacromolecules, including but not limited to Fc-containing proteins, antibodies, antibody fragments, antibody derivatives, fusion proteins, Fc-fusion proteins, receptor Fc-fusion proteins, trap proteins and mini-trap proteins. Nanoparticles also can be analyzed. Liquid chromatography (LC) systems also can be used with the A4F-MS systems according to the present inventions.

### BACKGROUND OF THE INVENTIONS

Asymmetrical flow field-flow fractionation is a useful technique for analyzing biomacromolecules and associated complexes (for example, protein aggregates and antibody-antigen assemblies). Size exclusion chromatography (SEC) and A4F separate biomacromolecules based on their hydrodynamic radius. However, A4F does not use a stationary phase and provides a gentler separation than SEC. Therefore, A4F is ideally suited for analyzing labile biomacromolecules and complexes by better preserving their native conformations and interactions. In the past, however, direct coupling of A4F with native MS (nMS) detection has not been successful, largely due to challenges in buffer/flow rate incompatibilities and insufficient MS sensitivity.

There have been published approaches combining hollow fiber and chip-type asymmetrical fractionation with mass spectrometry. Kim et al., J. Chromatog. A 1280: 92-97 (2013); Kim et al., Anal. Chem. 83: 8652-58 (2011); Reschliglian et al., Anal. Chem. 77: 47-56 (2005). These approaches are complex, however.

Accordingly, there exists a need for novel and improved A4F-nMS platforms that are amenable to commercially available instruments and parts, and have demonstrated successful application in characterizing therapeutic biomacromolecules and nanoparticles of differing and varying complexity and heterogeneity.

### SUMMARY OF THE INVENTIONS

The inventions provide systems and methods for analyzing proteins in a sample using asymmetrical flow field flow fractionation (A4F) and mass spectrometry (MS). The systems according to the inventions can perform methods according to the inventions. The methods according to the invention can comprise the steps of: (a) providing a liquid sample comprising proteins in an ammonium salt buffer; (b) fractionating the liquid sample by an A4F instrument to provide a fractionated liquid sample; and (c) sending a microflow of the fractionated liquid sample to a mass spectrometer for spectral analysis. The methods can further comprise characterizing the proteins based upon the spectral analysis. The microflow can be provided by using a tee to split the sample flow from the A4F instrument. The A4F instrument can have a short channel.

The proteins analyzed by the systems and methods can be Fc-containing proteins, such as antibodies, antibody fragments, including antibodies that lack Fv regions. The antibodies can be one or more of monoclonal antibodies and multi-specific antibodies.

The Fc-containing proteins also can be Fc-fusion proteins, such as receptor-Fc-fusion proteins and trap proteins.

The proteins analyzed by the systems and methods can be mini-trap proteins.

The proteins analyzed by the systems and methods can characterize protein complexes. The protein complexes can be homogeneous or heterogeneous. For example, the heterologous protein complexes can comprise any one or more of (1) antibodies and antigens, (2) antibody fragments and antigens, (3) antibody/antibody fragment and anti-antibody/anti-antibody fragment complexes (optionally including antigens), (4) receptor Fc-fusion proteins and ligands, (5) trap proteins and ligands, (6) mini-trap proteins and ligands and (7) any of complexes 1-6 above and antibodies/antibody fragments against proteins of 1-6 above.

The ammonium salt for use according to the inventions can be ammonium acetate. Preferably, the concentration of ammonium acetate can be selected from about: 25 mM to 500 mM, 50 mM to 450 mM, 75 mM to 400 mM, 100 mM to 350 mM, 100 mM to 300 mM, 100 mM to 250 mM, 100, 140 mM to 160 mM, 140 mM to 150 mM, 145 mM to 155 mM, 145 mM to 150 mM and less than or equal to 150 mM.

The microflow for use according to the invention can be selected from about: 0.1 μl/min to 10 μl/min, 0.5 μl/min to 10 μl/min, 0.6 μl/min to 9 μl/min, 0.65 μl/min to 8.5 μl/min, 0.75 μl/min to 8 μl/min, 0.85 μl/min to 7 μl/min, 1 μl/min to 6 μl/min, 1.25 μl/min to 5 μl/min, 1.5 μl/min to 4 μl/min, 1.5 μl/min to 3.5 μl/min, 1.75 μl/min to 3.0 μl/min, 1.75 μl/min to 2.75 μl/min, 1.75 μl/min to 2.50 μl/min, 1.8 μl/min to 2.25 μl/min, 1.9 l/min to 2.1 μl/min, 1.0 μl/min to 2.0 μl/min, 1.5 μl/min to 2.0 μl/min, 1.75 μl/min to 2.0 μl/min and less than or equal to 2 μl/min, such as 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 μl/min.

### BRIEF DESCRIPTION OF THE FIGURES

FIG. 1 schematically depicts an A4F instrument coupled to an MS instrument according to the inventions.

FIG. 2 schematically depicts in greater detail an A4F instrument coupled to an MS instrument according to the inventions.

FIG. 3 is a graph of the effect of different concentrations of ammonium acetate on protein recovery.

FIG. 4 depicts data from an A4F-UV analysis of a protein mixture having molecular weights ranging from 17 kilodaltons to 660 kilodaltons.

FIGS. 5A and 5B depict data of testing of four antibodies with similar molecular weights and different isoelectric points, and were used to compare the separations between the TIC (total ion chromatogram) of A4F-MS (FIG. 5A) and the TIC of SEC-MS (FIG. 5B).

FIG. 6A depicts data from liquid chromatography and A4F showing the separation of high molecular weight species of a monoclonal antibody. FIG. 6B depicts data from mass spectroscopy showing the separation of high molecular weight species of a monoclonal antibody.

FIG. 7A depicts TIC (total ion chromatogram) and (XIC). (extracted ion chromatogram). FIG. 7B depicts data showing that UHMR mass spectrometer can detect monomers, dimers, trimers and tetramers species.

### DETAIL DESCRIPTION OF THE INVENTIONS

Details of the inventions are further provided below.

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform, as is apparent from the teachings contained herein. The term permits standard variation as would be understood by those of ordinary skill in the art, and where ranges are provided, endpoints are included. For example, “about” can signify values either above or below the stated value in a range of approximately +/−10% or more or less depending on the ability to perform. Thus, this term encompasses values beyond those simply resulting from systematic error.

“Antibodies” (also referred to as “immunoglobulins”) are examples of proteins having multiple polypeptide chains and can have extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

Antibodies are often used as therapeutic biomolecules. An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRI, CDRI, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (for example, in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies), multispecifics (for example, IgG-ScFv, IgG-dab, ScFV-Fc-ScFV, tri-specific).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

“Fc-fusion proteins” are a type of fusion protein and comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci. USA 88: 10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which can comprise a hinge region followed by a CH2 and CH3 domain of an immunoglobulin. The Fc-fusion protein can contain two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-1R1 extracellular region fused to Fc of hlgGI; see U.S. Pat. No. 6,927,044, or a VEGF Trap (for example, Aflibercept, which contains the Ig domain 2 of the VEGF receptor FltI fused to the Ig domain 3 of the VEGF receptor FlkI fused to Fc of hlgG1 See U.S. Pat. Nos. 7,087,411 and 7,279,159.

Fc-containing proteins, such as antibodies, can comprise modifications in immunoglobulin domains, including where the modifications affect one or more effector function of the binding protein (for example, modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V259I), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

“Polypeptide” and “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

“Protein of interest” or “polypeptide of interest” can have any amino acid sequence, and includes any protein, polypeptide, or peptide, and derivatives, components, domains, chains and fragments thereof. Included are, but not limited to, viral proteins, bacterial proteins, fungal proteins, plant proteins and animal (including human) proteins. Protein types can include, but are not limited to, antibodies, bi-specific antibodies, multi-specific antibodies, antibody chains (including heavy and light), antibody fragments, Fv fragments, Fc fragments, Fc-containing proteins, Fc-fusion proteins, receptor Fc-fusion proteins, receptors, receptor domains, trap and mini-trap proteins, enzymes, factors, repressors, activators, ligands, reporter proteins, selection proteins, protein hormones, protein toxins, structural proteins, storage proteins, transport proteins, neurotransmitters and contractile proteins. Derivatives, components, chains and fragments of the above also are included. The sequences can be natural, semi-synthetic or synthetic. Proteins of interest and polypeptides of interest are encoded by “genes of interest,” which also can be referred to as “polynucleotides of interest.”

“Protein complexes” comprise protein molecules bound by natural forces, including electrostatic interactions, hydrogen bonding, van der Waals forces, hydrophilic interactions, and hydrophobic interactions. A protein complex is homologous if the protein molecules of the complex are the same, such as multimers of a particular antibody. A protein complex is heterologous if the protein molecules are different, such as different types of antibodies/antibody fragments bound together, the same or different types of antibodies/antibody fragments bound to one or more types of antigens, or the same or different ligands bound to proteins comprising receptor moieties. Heterologous complexes includes complexes that are not homologous.

An “Extracted Ion Chromatogram” (XIC) is created by plotting the intensity of the signal observed at chosen mass-to-charge value or series of values in a series of mass spectra recorded as a function of retention time.

A “Total Ion Chromatogram” (TIC) is a chromatogram created by summing up intensities of all mass spectral peaks belonging to the same scan.

All numerical limits and ranges set forth herein include all numbers or values thereabout or there between of the numbers of the range or limit, and include the end-points. The ranges and limits described herein expressly denominate and set forth all integers, decimals and fractional values defined and encompassed by the range or limit. The ranges and limits described herein expressly denominate and set forth all integers, decimals and fractional values defined and encompassed by the range or limit. Thus, a recitation of ranges of values herein are merely intended to serve as an efficient method of referring individually to each separate value falling within the range, unless otherwise indicated herein, and each separate value is incorporated into the specification as if it were individually recited herein.

### DETAILED DESCRIPTION

The inventions can be employed in the characterization and production of biological and pharmaceutical products, including next-generation versions of existing biological and pharmaceutical products produced in cell culture. A wide range of protein-based therapeutics, such as monoclonal antibody-based therapeutics, can be analyzed and produced according to the inventions. For example, cells comprising requisite DNA sequences encoding antibodies, including but not limited to the antibodies identified below, can be grown in culture according the present inventions. The inventions can be used to analyze aggregates of proteins, such as antibodies, antibody-drug conjugates, antibody-antigen assemblies, complexes of ligands and receptors, including receptor moieties on receptor-Fc fusion proteins and min-trap proteins, lipid nanoparticles, RNA and DNA.

The following identifies and describes proteins made in cell culture that can be produced according to the present inventions. Cells comprising the requisite DNA encoding these proteins can be cultured for production according to the present inventions.

For example, for antibody production, the inventions are amendable for research and production use for diagnostics and therapeutics based upon all major antibody classes, namely IgG, IgA, IgM, IgD and IgE. IgG is a preferred class, and includes subclasses IgG1 (including IgG1λ and IgG1κ), IgG2, IgG3, and IgG4. Further antibodies include a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. The antibody can be an IgG1 antibody. The antibody can be an IgG2 antibody. The antibody can be an IgG4 antibody. The antibody can be a chimeric IgG2/IgG4 antibody. The antibody can be a chimeric IgG2/IgG1 antibody. The antibody can be a chimeric IgG2/IgG1/IgG4 antibody. Derivatives, components, domains, chains and fragments of the above also are included.

Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, an antibody derivative, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. The antibody can be an IgG1 antibody. The antibody can be an IgG2 antibody. The antibody can be an IgG4 antibody. The antibody can be a chimeric IgG2/IgG4 antibody. The antibody can be a chimeric IgG2/IgG1 antibody. The antibody can be a chimeric IgG2/IgG1/IgG4 antibody.

The antibody can be selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-DII4 antibody, an anti-Angiopoetin-2 antibody (for example an anti-ANG2 antibody as described in U.S. Pat. No. 9,402,898), an anti-Angiopoetin-Like 3 antibody (for example an anti-AngPtl3 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-C5 antibody as described in U.S. Pat. Appln. Pub. No US2015/0313196A1), an anti-TNF antibody, an anti-epidermal growth factor receptor antibody (for example an anti-EGFR antibody as described in U.S. Pat. No. 9,132,192 or an anti-EGFRvlll antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat. No. 8,871,209 or 9,260,515), an anti-Glucagon Receptor (for example anti-GCGR antibody as described in U.S. Pat. Appln. Pub. Nos. US2015/0337047A1 or US2016/0075778A1), an anti-VEGF antibody, an anti-IL1R antibody, an interleukin 4 receptor antibody (for example, an anti-IL4R antibody as described in U.S. Pat. Appln. Pub. No. US2014/0271681A1 or U.S. Pat. No. 8,735,095 or 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example, anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088297A1 and US20150266966A1, and in U.S. Application No. 62/222,605), an anti-Cluster of differentiation 20 (for example an anti-CD20 antibody as described in U.S. Pat. Appln. Pub. Nos. US2014/0088297A1 and US20150266966A1, and in U.S. Pat. No. 7,879,984), an anti-CD19 antibody, an anti-CD28 antibody, an anti-Cluster of Differentiation 48 (for example anti-CD48 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Activin A antibody. The bispecific antibody can be selected from the group consisting of an anti-CD3×anti-CD20 bispecific antibody (as described in U.S. Pat. Appln. Pub. Nos. US2014/0088297A1 and US20150266966A1), an anti-CD3×anti-Mucin 16 bispecific antibody (for example, an anti-CD3×anti-Muc16 bispecific antibody), and an anti-CD3×anti-Prostate-specific membrane antigen bispecific antibody (for example, an anti-CD3×anti-PSMA bispecific antibody). See also U.S. Patent Publication No. US 2019/0285580 A1. Also included are a Met×Met antibody, an agonist antibody to NPR1, an LEPR agonist antibody, a BCMA×CD3 antibody, a MUC16×CD28 antibody, a GITR antibody, an IL-2Rg antibody, an EGFR×CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured according to the inventions. Exemplary antibodies include Alirocumab, Atoltivimab, Maftivimab, Odesivimab, Odesivivmab-ebgn, Casirivimab, Imdevimab, Cemiplimab and Cemiplimab-rwlc (human IgG4 monoclonal antibody that binds PD-1), Dupilumab (human monoclonal antibody of the IgG4 subclass that binds to the IL-4R alpha (a) subunit and thereby inhibits Interleukin 4 (IL-4) and Interleukin 13 (IL-13) signaling), Evinacumab, Evinacumab-dgnb, Fasinumab, Fianlimab, Garetosmab, Itepekimab Nesvacumab, Odrononextamab, Pozelimab, Sarilumab, Trevogrumab, and Rinucumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product that is shown that may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to an reference product already approved in Japan. In India, biosimilars currently are referred to as “similar biologics,” and refer to a similar biologic product is that which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production and analysis of biosimilars and its synonyms under these and any revised definitions can be undertaken according to the inventions.

The inventions advantageously couple available A4F systems with mass spectrometers, such as native mass spectrometry. US 2021/0239661 A1 publication discloses mass spectrometry systems for use according to the inventions taught herein. As disclosed herein, a Wyatt A4F Eclipse and a Wyatt short separation channel can be coupled to a Thermo Q-Exactive UHMR, for example. Liquid chromatography (LC) systems, such as an Agilent Infinite II LC system, can be used for solvent delivery, sample pick up and loading and UV signal acquisition. Other LC, A4F and MS systems are amenable for use according to the inventions.

An A4F instrument should be operated at low pressure. In order to keep the A4F-MS system pressure within an optimal range, it is important to organize all instruments, including the LC, A4F, separation channel, and MS in a compact and efficient manner to minimize the length of tubing.

After A4F separation, partial sample flow is directed to the MS through the “Fraction Collector” port on the A4F. Output flow rate from A4F to MS could affect system pressure. It should be carefully optimized to keep the system pressure within an operable range. The output flow rate is preferably as described below.

The microflow for use according to the invention can be selected from about: 0.1 μl/min to 10 μl/min, 0.5 l/min to 10 μl/min, 0.6 μl/min to 9 μl/min, 0.65 μl/min to 8.5 l/min, 0.75 μl/min to 8 μl/min, 0.85 μl/min to 7 μl/min, 1 μl/min to 6 μl/min, 1.25 μl/min to 5 μl/min, 1.5 μl/min to 4 μl/min, 1.5 l/min to 3.5 μl/min, 1.75 μl/min to 3.0 μl/min, 1.75 μl/min to 2.75 μl/min, 1.75 μl/min to 2.50 μl/min, 1.8 μl/min to 2.25 μl/min, 1.9 μl/min to 2.1 μl/min, 1.0 μl/min to 2.0 μl/min, 1.5 μl/min to 2.0 μl/min, 1.75 μl/min to 2.0 μl/min and less than or equal to 2 μl/min, such as 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 μl/min.

A stainless-steel micro-tee was applied to split the flow from A4F to MS from analytical flow (about 0.2 ml/min) to microflow (sub μl/min) for MS detection.

A Thermo UHMR mass spectrometer can be equipped with a NewOmics™ MnESI microflow ion Source was used for native MS analysis. To achieve nanoelectrospray ionization (NSI) (a type of ESI, electrospray ionization) with microflow, a Newomics Microfabricated Monolithic Multi-nozzle (M3) emitter was applied.

NSI permits this native MS platform to achieve superior sensitivity and tolerance for up to 600 mM of ammonium salts. Preferably, the concentration of ammonium acetate can be selected from about: 25 mM to 500 mM, 50 mM to 450 mM, 75 mM to 400 mM, 100 mM to 350 mM, 100 mM to 300 mM, 100 mM to 250 mM, 100, 140 mM to 160 mM, 140 mM to 150 mM, 145 mM to 155 mM, 145 mM to 150 mM and less than or equal to 150 mM.

Via desolvation gas modification, the inventions can readily achieve online charge reduction native MS, which facilitates analysis of labile and heterogeneous molecules, including biomacromolecules and nanoparticles. The aspects of the inventions are described in further detail below in relation to the figures.

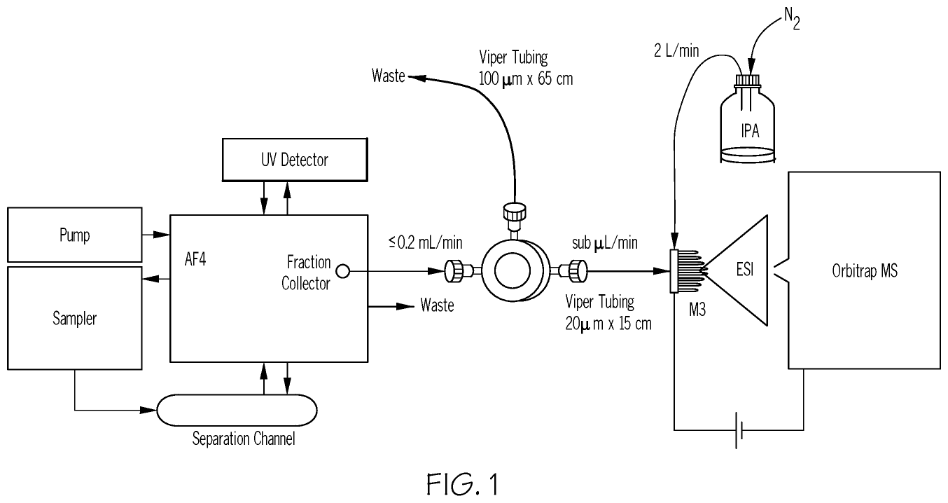

FIG. 1 depicts a simplified schematic of an AF4 (A4F)-MS platform. A pump is operatively connected to and delivers fluid to the AF4 instrument, which is in turn operatively connected to a sampler, which contains biomacromolecules or nanoparticles. The sampler is operatively connected to separation channel, which receives sample-containing fluid. The sample-containing fluid is separated by the separation channel and then sent to the AF4 instrument, and then the AF4 instrument sends the majority of the fluid to the UV detector and remainder to the mass spectrometer (MS). The flow is preferably less than or equal to 0.2 milliliters per minute.

Sample-containing fluid is sent to the MS, but the majority of the fluid is sent to waste collection. A sub microliter flow of the sample-containing fluid is combined with nitrogen gas that has been pumped through isopropyl alcohol (IPA), preferably at 2 liters per minute, and then in turn is sent to a multinozzle emitter (M3) for electrospray ionization (ESI) for mass spectroscopy.

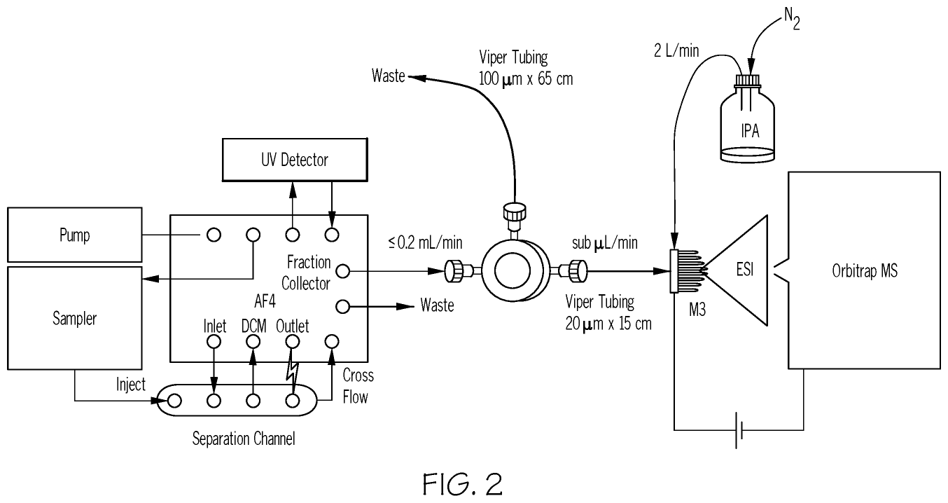

FIG. 2 provides greater detail on the AF4 instrument and the separation channel. Inlet, dilution control module (DCM), outlet and crossflow corresponding ports are contained in the AF4 instrument and the separation channel.

The inventions are further described by the following Examples, which are illustrative of the many aspects of the invention, but do not limit the inventions in any manner.

### Example 1

A Wyatt A4F Eclipse instrument with a short separation channel was used for sample separation. An Agilent Infinite 1260 II LC system was used for solvent delivery, sample loading, and UV signal acquisition. Ammonium acetate buffer was used as mobile phase. After A4F separation, partial sample flow was directed to the MS through the “Fraction Collector” port on the A4F. A stainless-steel micro-tee was then used to split the sample flow to a microflow (about 2 μl/min) for MS detection. A Thermo UHMR mass spectrometer equipped with a NewOmics™ MnESI microflow ion Source was used for native MS analysis. A protein standard mix, mAbs with different pls, mAb aggregates, and an antigen-antibody mixture were used for A4F-MS platform performance evaluation. See FIGS. 1 and 2. Other types and sources of A4F, LC and MS instruments can be used according to the inventions.

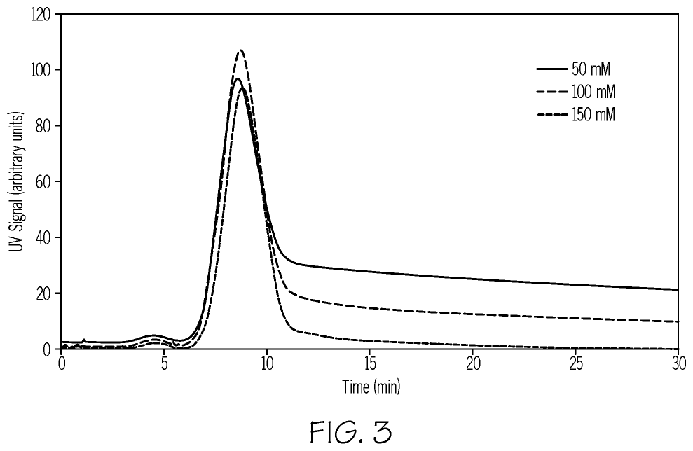

Ammonium acetate of different concentrations (50 mM to 150 mM) were first evaluated as MS-compatible mobile phases for A4F separation of protein samples. See FIG. 3 (using a NIST reference antibody). Interestingly, despite being a stationary phase-free method, A4F exhibited suboptimal recovery when lower salt concentration of mobile phase was used. Considering MS sensitivity is inversely correlated to salt concentration, a final mobile phase containing 150 mM ammonium acetate was selected to allow both maximal protein recovery and sensitive native MS detection. Following, other instrument parameters, including flow rates and flow splitting ratios at both channel outlet and fractionation port, and connection designs to MS, were also carefully optimized to maintain A4F resolution during MS detection.

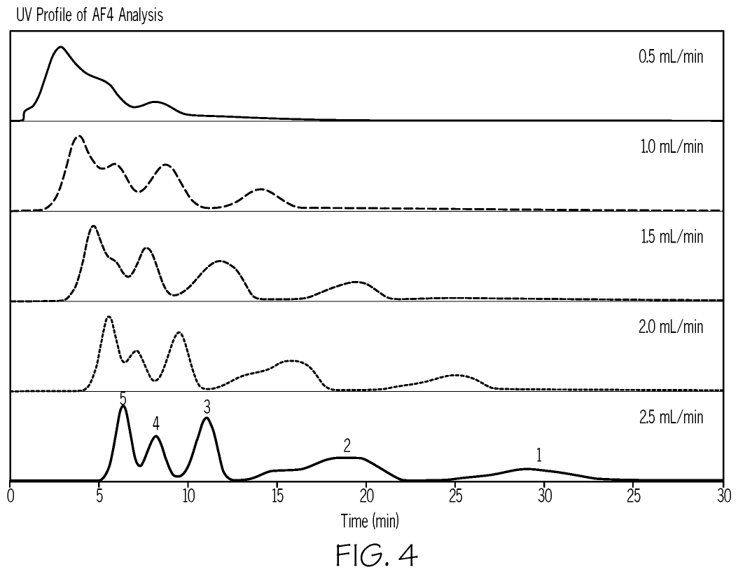

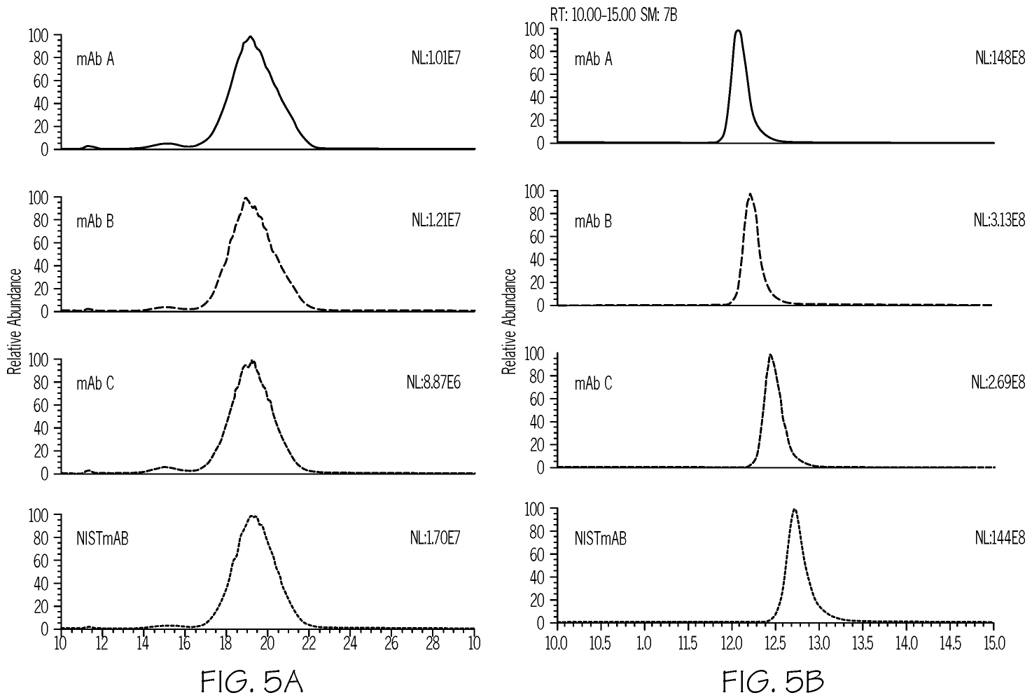

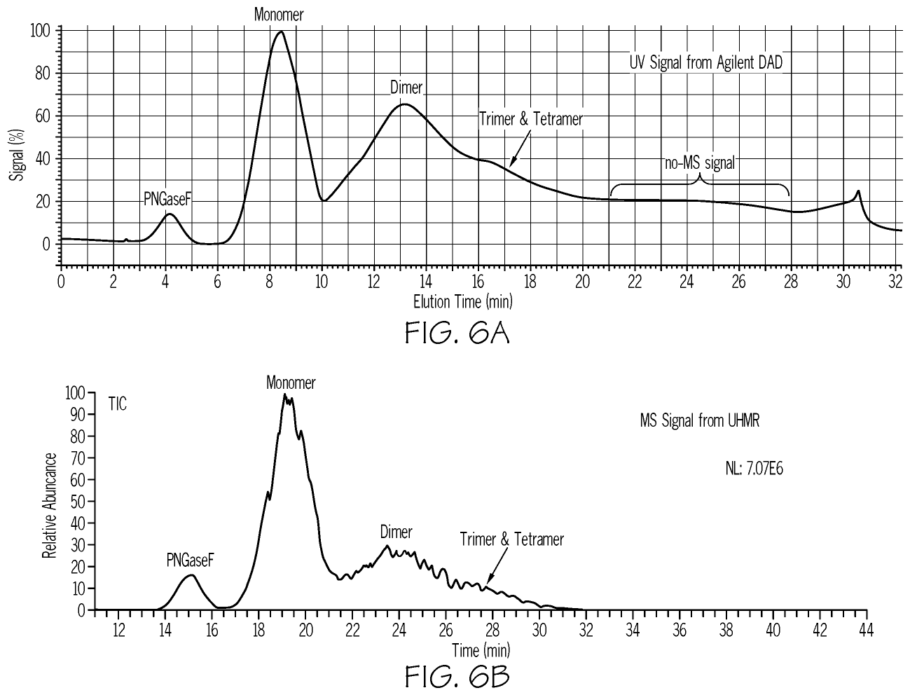

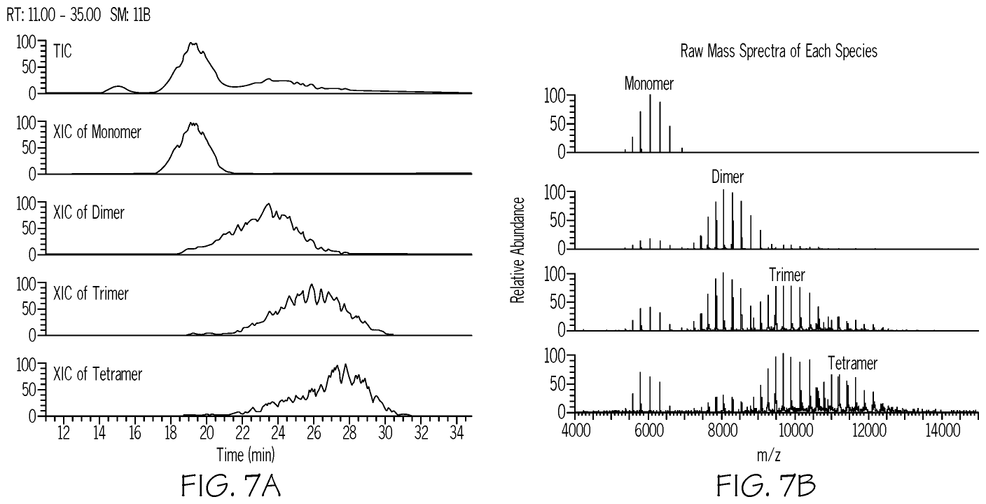

Performance was evaluated using different protein combinations, including but not limited to:
Example 2—a protein standard mix containing proteins with MW ranging between 17 kDa to 660 kDa (FIG. 4);
Example 3—mAb molecules with pI values ranging between 6.5 to 8.9 (FIGS. 5A and 5B); and
Example 4—an enriched mAb HMW sample containing size variants from monomer to pentamer (FIGS. 6A, 6B, 7A and 7B).

Quantitative performance was evaluated and demonstrated excellent linearity and reproducibility.

### Example 2

An A4F-UV analysis was conducted with a protein mixture having molecular weights ranging from 17 kilodaltons to 660 kilodaltons:

| Identification | | Molecular Weight |
|---|---|---|
| number in FIG. 4 | Protein | (kilodaltons) |
| 1 | Thyroglobulin | 660 |
| 2 | IgG | 150 |
| 3 | Bovine serum albumin | 66 |
| 4 | PNGaseF | 34 |
| 5 | Myoglobin | 17 |

In conducting the A4F, 150 mM ammonium acetate was used as the solvent. The channel flow was 1 ml/minute, and the cross flows ranging from 0.5 ml/minute (top) to 2.5 ml/minute (bottom) are shown in FIG. 4. This tests established that A4F can separate samples with a wide range of molecular weight distributions. Higher cross flow rates resulted in better separation.

### Example 3

A4F-MS and size exclusion chromatography (SEC)-MS was conducted with four monoclonal antibodies having different isoelectric points (pI):

| Monoclonal AB | Average Molecular Weight | Isoelectric point |
|---|---|---|
| mAb A | 144,656.1 | 6.5 |
| mAb B | 143,364.9 | 7.5 |
| mAb C | 146,256.2 | 8.9 |
| NISTmAb | 145,869.4 | 8.8 |

The loading amount was 10 μg for each antibody, and the solvent was 150 mM ammonium acetate.

The four antibodies had similar molecular weights and different isoelectric points, and were used to compare the separations between the TIC of A4F-MS (FIG. 5A) and the TIC of SEC-MS (FIG. 5B). The antibodies with different isoelectric points elute at practically the same time, which means that A4F can eliminate the effects of isoelectric points in separation. See FIG. 5A. In contrast, higher isoelectric point values correlate with longer elution times with SEC. See FIG. 5B.

Thus, A4F-MS has the advantage of less influenced by different isoelectric points, and thus superior for weight-based separation.

### Example 5

Parallel analyses using native (n)SEC-MS can be performed to compare the two methods and demonstrate certain unique advantages offered by A4F-MS. According to the inventions, it is expected that A4F will offer (1) superior separation at higher MW region (for example, mAb tetramer and above), (2) superior recovery for proteins that showed enhanced secondary interaction with SEC column matrix, and 3) the ability to better preserve labile complexes during separation. Antibody-antigen mixture consisting of complexes with different binding stoichiometry with A4F-nMS also can be undertaken, and then compared to nSEC-MS.

Turning to FIGS. 6A and 6B, an Agilent LC was coupled to an A4F for sample injection flow delivery and UV-data collection. The protein analyzed was a CD20×CD3 bispecific antibody. The antibody sample contained antibody monomers, dimers and very high molecular weight species in a ratio of 1:1:3. The sample was treated with PNGaseF, which removes N-linked oligosaccharides from glycoproteins.

The sample loading was 12 μg (4 μl). The mobile phase comprised 150 mM ammonium acetate. See FIG. 3, The peaks depicted in FIGS. 6A and 6B are consistent. FIG. 6A depicts to UV signal of size profile analyzed by A4F. FIG. 6B shows the MS signal for the same samples as in FIG. 6A.

FIGS. 7A and 7B depict additional analysis on the same sample sources as FIGS. 6A and 6B. The first panel of FIG. 7A (TIC) displays the same data as FIG. 6B (MS data for the CD20×CD3 antibody) The second to fifth panels show the extracted signal (XIC) of the monomer, dimer, trimer and tetramer species. FIG. 7B depicts the mass spectrum of each species from the second to fifth panels of FIG. 7A, and establishes the advantages and power of the present inventions.

It is to be understood that the description, specific examples and data are given by way of illustration and are not intended to limit the present invention. Various changes and modifications within the present inventions, including combining any and all teachings in whole and in part, will become apparent to the skilled artisan from the discussion, disclosure and data contained herein, and thus are considered part of the inventions.

1. A method for analyzing proteins in a sample using asymmetrical flow field flow fractionation (A4F) and mass spectrometry (MS), wherein the method comprises the steps of:
(a) providing a liquid sample comprising proteins in an ammonium salt buffer;
(b) fractionating the liquid sample by an A4F instrument to provide a fractionated liquid sample; and
(c) sending a microflow of the fractionated liquid sample to a mass spectrometer for spectral analysis.

2. The method according to claim 1, further comprising characterizing the proteins based upon the spectral analysis.

3. The method according to claim 1, wherein the microflow is provided by using a tee to split the sample flow from the A4F.

4. The method according to claim 3, wherein A4F instrument has a short channel.

5. The method according to claim 1, wherein the proteins are Fc-containing proteins.

6. The method according to claim 5, wherein the Fc-containing proteins are antibodies.

7. The method according to claim 6, wherein the antibodies are monoclonal antibodies.

8. The method according to claim 6, wherein the monoclonal antibodies are multi-specific antibodies.

9. The method according to claim 5, wherein the Fc-containing proteins are antibody fragments.

10. The method according to claim 5, wherein the Fc-containing proteins are antibody fragments that lack Fv regions.

11. The method according to claim 5, wherein the Fc-containing proteins are Fc-fusion proteins.

12. The method according to claim 11, wherein the Fc-containing proteins are receptor Fc-fusion proteins.

13. The method according to claim 12 wherein the receptor Fc-fusion proteins are trap proteins.

14. The method according to claim 1, wherein the proteins are mini-trap proteins.

15. The method according to claim 1, wherein the method can characterize protein complexes.

16. The method according to claim 15, wherein the protein complexes are:
homogeneous; or
heterogeneous.

17. (canceled)

18. The method according to claim 15, wherein the protein complexes comprise:
antibodies and antigens;
antibody fragments and antigens;
receptor Fc-fusion proteins and ligands; or
mini-trap proteins and ligands.

19.-21. (canceled)

22. The method according to claim 1, wherein the ammonium salt is ammonium acetate.

23. The method according to claim 1, wherein the ammonium salt is:
present at a concentration of 25 mM to 500 mM;
present at a concentration of 50 mM to 450 mM;
present at a concentration of 75 mM to 400 mM;
present at a concentration of 100 mM to 350 mM;
present at a concentration of 100 mM to 300 mM;
present at a concentration of 100 mM to 250 mM;
present at a concentration of 100 mM to 200 mM;
present at a concentration of 125 mM to 175 mM;
present at a concentration of 140 mM to 160 mM;
present at a concentration of 140 mM to 150 mM;
present at a concentration of 145 mM to 150 mM;
present at a concentration of 145 mM to 155 mM; or
present at a concentration of 150 mM.

24.-35. (canceled)

36. The method according to claim 1, wherein the microflow is:
0.5 μl/min to 10 μl/min;
0.75 μl/min to 8 μl/min;
1 μl/min to 6 μl/min;
1.25 μl/min to 5 μl/min;
1.5 μl/min to 4 μl/min;
1.5 μl/min to 3.5 μl/min;
1.75 μl/min to 3.0 μl/min;
1.75 μl/min to 2.75 μl/min;
1.75 μl/min to 2.50 μl/min;
1.8 μl/min to 2.25 μl/min;
1.9 μl/min to 2.1 μl/min;
1.0 μl/min to 2 μl/min;
1.5 μl/min to 2 μl/min;
1.75 μl/min to 2 μl/min; or
2 μl/min.

37.-50. (canceled)

51. A system capable of performing a method according to claim 1 and the description contained herein.
